(19) United States
(12) Patent Application Publication
(10) Pub. No.: US 2006/0200156 A1
(43) Pub. Date: Sep. 7, 2006

(54) Spinal docking system, spinal docking device, and methods of spinal stabilization

(76) Inventors: Jamal Taha, Cincinnati, OH (US); Gary Kraus, Houston, TX (US)

Correspondence Address: DINSMORE & SHOHL LLP, ONE DAYTON CENTRE, ONE SOUTH MAIN STREET, SUITE 1300, DAYTON, OH, 45402-2023, US

(21) Appl. No.: 11/326,592

(22) Filed: Jan. 5, 2006

Related U.S. Application Data
(60) Provisional application No. 60/641,404, filed on Jan. 5, 2005.
(60) Provisional application No. 60/641,408, filed on Jan. 5, 2005.

(51) Int. Cl. A61F 5/00

(52) U.S. Cl. 606/86

(57) ABSTRACT

The present invention relates to spinal docking systems, spinal docking devices, and methods of spinal stabilization. According to one embodiment of the present invention, a spinal docking system comprises a spinal docking device, a docking device coupling mechanism, and a surgical instrument guide. In accordance with the present invention, an alternative embodiment of a spinal docking system comprises a spinal docking device and a surgical instrument guide. In accordance with yet another embodiment of the present invention, a spinal docking device comprises a concave end and a longitudinal face. Another embodiment of the present invention relates a method of stabilizing a pedicle.

### CROSS-REFERENCE TO RELATED APPLICATIONS

This application claims the benefit of U.S. Provisional Application Ser. Nos. 60/641,404 (JAM 0004 MA), filed Jan. 5, 2005 and 60/641,408 (JAM 0005 MA), filed Jan. 5, 2005.

### BACKGROUND OF THE INVENTION

The present invention relates to spinal docking systems, spinal docking devices, and methods of spinal stabilization.

### BRIEF SUMMARY OF THE INVENTION

According to the present invention, a variety of spinal docking systems, spinal docking devices, and methods of spinal stabilization are provided. In accordance with one embodiment of the present invention, a spinal docking system comprises a spinal docking device, a docking device coupling mechanism, and a surgical instrumentation guide. The spinal docking device comprises a concave end, which comprises a degree of concavity that is characterized by dimensions of width and depth that are sufficient to allow the concave end to straddle an aspect of the vertebral column that has a lateral dimension at least about half as large as a lateral dimension of a transverse process of the vertebral column. The docking device coupling mechanism is configured to secure the spinal docking device to the surgical instrumentation guide and to allow movement of the surgical instrumentation guide relative to the spinal docking device. This surgical instrumentation guide is configured to guide surgical instrumentation to an aspect of the vertebral column that is displaced from the straddled aspect of the vertebral column.

In accordance with another embodiment of the present invention, a spinal docking system comprises a spinal docking device and a surgical instrumentation guide. The spinal docking device comprises a concave end and one or more longitudinal faces where the concave end comprises a degree of concavity that is characterized by dimensions of width and depth that are sufficient to allow the concave end to straddle an aspect of the vertebral column that has a lateral dimension at least about half as large as a lateral dimension of a transverse process of the vertebral column. The longitudinal faces of the spinal docking device are secured to the surgical instrumentation guide, which is configured to guide surgical instrumentation to said straddled aspect of the vertebral column.

In accordance with yet another embodiment of the present invention, a spinal docking device comprises a concave end and one or more longitudinal faces. The concave end comprises a degree of concavity that is characterized by dimensions of width and depth that are sufficient to allow the concave end to straddle an aspect of the vertebral column that has a lateral dimension at least about half as large as a lateral dimension of a transverse process of the vertebral column. The longitudinal face comprises a channeled depression to guide surgical instrumentation to the straddled aspect of the vertebral column or to an aspect of the vertebral column that is displaced from the straddled aspect of the vertebral column.

In accordance with yet another embodiment of the present invention, a method of stabilizing a pedicle is presented. The method comprises providing a spinal docking system that comprises a spinal docking device, a docking device coupling mechanism, and a surgical instrumentation guide. This spinal docking device comprises a concave end with a degree of concavity that is characterized by dimensions of width and depth that are sufficient to allow the concave end to straddle a transverse process of a vertebral column. The method further comprises positioning the concave end and the surgical instrumentation guide, which is adjustably secured to the spinal docking device by the docking device coupling mechanism, on a transverse process such that the spinal docking device and the surgical instrumentation guide are aligned substantially perpendicularly to the transverse process. Thereafter, the method comprises guiding through the surgical instrumentation guide a surgical instrument in the general direction of the transverse process and operating the surgical instrument so as to enable the insertion of spinal stabilization hardware medial to the transverse process and into the pedicle.

Accordingly, it is an object of the present invention to provide improved spinal docking systems, spinal docking devices, and methods of spinal stabilization. Other objects of the present invention will be apparent in light of the description of the invention embodied herein.

### BRIEF DESCRIPTION OF THE SEVERAL VIEWS OF THE DRAWINGS

The following detailed description of specific embodiments of the present invention can be best understood when read in conjunction with the following drawings, where like structure is indicated with like reference numerals and in which:

FIG. 1 is a schematic illustration of a spinal docking system.

FIG. 2 is an illustration of a spinal docking device with a concave end resting on a transverse process of a vertebral column viewed laterally.

FIG. 3 is a schematic illustration of a spinal docking device secured to a surgical instrument guide by a docking device coupling mechanism held within a vertical slot on the spinal docking device.

FIG. 4A is a schematic illustration of a centered, curved embodiment of the concave end of a spinal docking device.

FIG. 4B is a schematic illustration of a centered, multi-angular embodiment of the concave end of a spinal docking device and a surgical instrument guide centered on a longitudinal face of the spinal docking device.

FIG. 4C is a schematic illustration of a centered, angled embodiment of the concave end of a spinal docking device and a surgical instrument guide offset from the longitudinal axis of a longitudinal face of the spinal docking device.

FIG. 4D is a schematic illustration of another centered, multi-angular embodiment of the concave end of a spinal docking device.

FIG. 4E is a schematic illustration of a curved concave end offset from the longitudinal axis of a spinal docking device.

### DETAILED DESCRIPTION

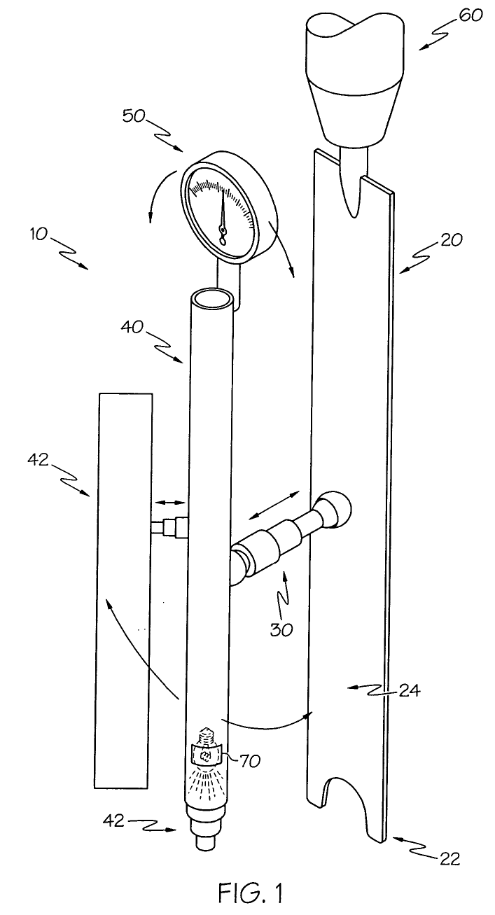

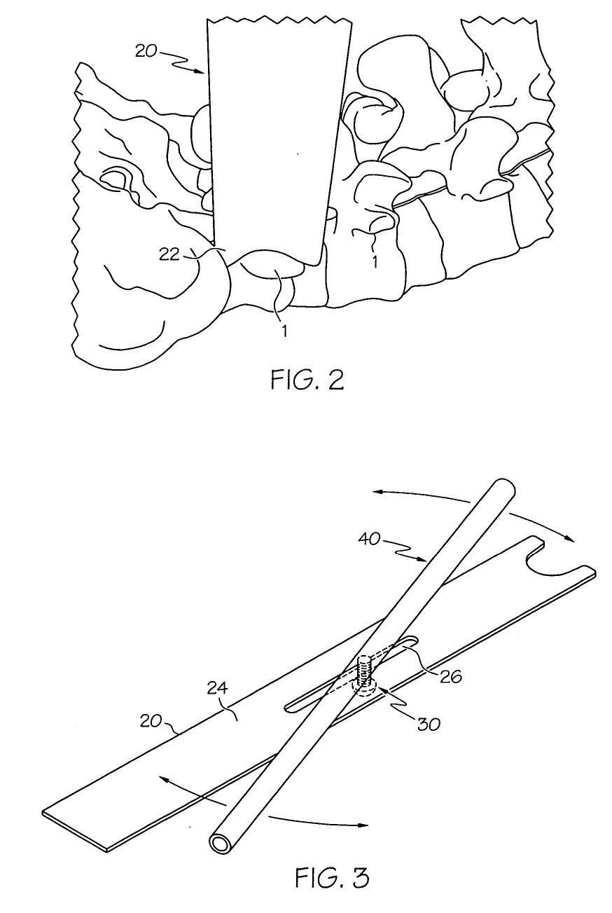

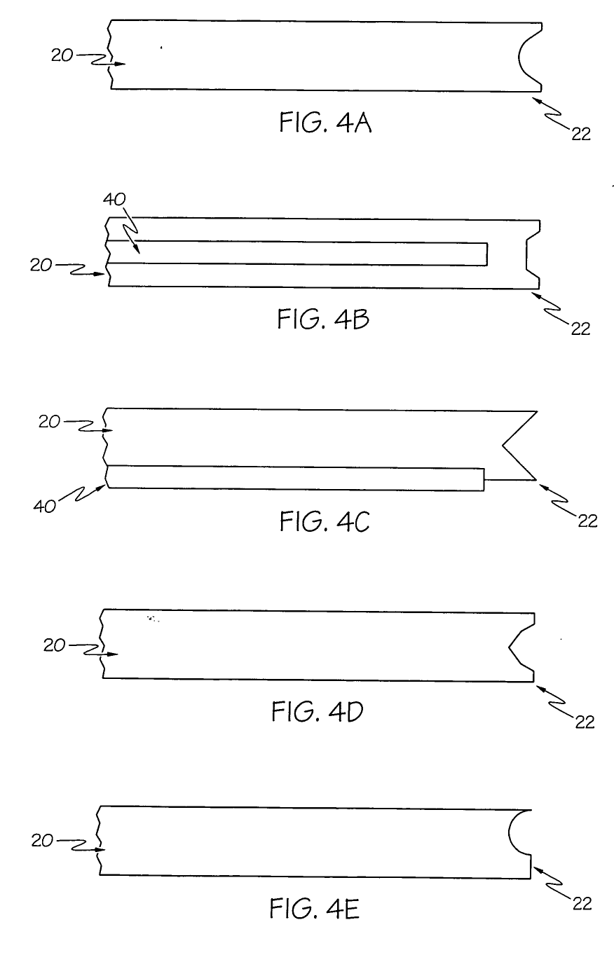

A spinal docking system 10 is illustrated schematically FIG. 1. This spinal docking system 10 generally comprises a spinal docking device 20, a docking device coupling mechanism 30, and a surgical instrument guide 40. Referring to FIG. 1, the spinal docking device 20 typically comprises a concave end 22 and a longitudinal face 24. This concave end comprises a degree of concavity that is characterized by dimensions of width and depth sufficient to allow the concave end to straddle an aspect of the vertebral column having a lateral dimension at least about half as large as a lateral dimension of a transverse process 1 of the vertebral column. Thereby, the concave end 22 can be configured to self-locate and self-stabilize on the transverse process 1 or other variously sized aspects of the vertebral column, including aspects that are larger than a transverse process 1, roughly the same size as a transverse process 1, or smaller than a transverse process 1.

The surgical instrument guide 40, meanwhile, generally is configured to guide surgical instrumentation to an aspect of the vertebral column displaced from that straddled aspect of the vertebral column. For purposes of this application, straddle means to closely overlap, to some degree, the two lateral ends of an aspect of a vertebral column, such as a transverse process 1, such that the concave end 22 rests on that aspect of the vertebral column in a stabile manner. The aspects of the vertebral column to which the spinal docking system may be applied includes any area of the vertebral column, such as, but not limited to, a transverse process, pedicle, facet, or spinous process.

The spinal docking device 20, the docking device coupling mechanism 30, and the surgical instrumentation guide 40 may be configured such that the straddled aspect and the displaced aspect comprise respective portions of a common aspect of the vertebral column. For example, the straddled aspect and the displaced aspect comprise respective portions of a common aspect of the vertebral column when they both occupy respective portions of a transverse process 1. In addition, the spinal docking device 20, the docking device coupling mechanism 30, and the surgical instrumentation guide 40 may be configured such that the straddled aspect and the displaced aspect comprise different aspects of the vertebral column. For example, the straddled aspect may comprise a portion of a transverse process while the displaced aspect comprises a portion of the adjacent facet and pedicle complex. Therefore, when the straddled aspect comprises a portion of a pedicle, transverse process, facet, spinous process, or other spinal structure of the vertebral column, the displaced aspect comprises a portion of a pedicle, transverse process, facet, spinous process, or other spinal structure of the vertebral column that is different from the portion comprised by the straddled aspect.

The spinal docking device 20 is generally configured such that this concave end 22 is provided in a curved, angular, multi-angular, or any combination thereof, configuration and is either centered or offset from a longitudinal axis of the spinal docking device. For example, the concave end may be provided in a circular, triangular, hexagonal, or other configuration. This concave end 22 may also be provided with clamping hardware that is configured to clamp the concave end 22 onto the straddled aspect of the vertebral column. Furthermore, the spinal docking device 20 may also be configured such that the concave end 20 comprises a dynamic cuff that is configured to adjust the degree of concavity. This dynamic cuff is configured to allow the spinal docking device to clamp and adhere to the straddled aspect of the vertebral column. It is contemplated that the dynamic cuff may take a variety of suitable forms including, but not limited to, a crank device, similar to that utilized in an adjustable wrench, to adjust the degree of concavity present in the concave end 22. Alternatively, the dynamic cuff may be configured to comprise a screw mechanism that adjusts the degree of concavity as said screw is tightened or loosened.

The docking device coupling mechanism 30 generally is configured to secure the spinal docking device 20 to the surgical instrumentation guide 40. This docking device coupling mechanism 40 typically is configured to allow movement of the surgical instrumentation guide relative to the spinal docking device and may be configured in lengths varying from very short to somewhat longer, as illustrated in FIG. 1. More specifically, the docking device coupling mechanism 40 generally comprises a device or assembly that is configured to allow movement of the surgical instrumentation guide 40 relative to the spinal docking device 20 in a plurality of degrees of freedom. For example, the device or assembly is configured to allow more than one concurrent pitch and yaw movements of the surgical instrumentation guide 40 along vertical, horizontal, tilt, and rotational axes. For example, and not by way of limitation, this device or assembly may be a hinge, fulcrum, ball socket, or other similarly related device or assembly. In addition, as shown in FIG. 3, to provide a greater range of movement of the surgical instrument guide 40, the spinal docking device 20 may be configured to define a vertical slot 26, or other track, to allow movement of the surgical instrumentation guide 40 along the longitudinal face 24 of the spinal docking device. The docking device coupling mechanism 30 illustrated in FIG. 3 may be a bolt, screw, or any other device or mechanism that allows for, but not necessarily limited to, sliding and swiveling movements of the surgical instrument guide 40 along the longitudinal face 24 of the spinal docking device 20. In addition, the docking device coupling mechanism 30 may further comprise hardware configured to indicate the positioning of the surgical instrumentation guide 40 relative to the spinal docking device 20 or relative to a plumb line and the line of gravity. An example of such hardware is a calibrated arc system or other similar device or system that measures and indicates the position of the surgical instrument guide 40. The docking device coupling mechanism 30 may further comprise a locking mechanism so as to fix the surgical instrument guide 40 in a position selected by a user of the spinal docking system 10. The spinal docking system 10 may be configured to comprise a plurality of docking device coupling mechanisms 30.

As mentioned above, the surgical instrumentation guide 40 generally is configured to guide surgical instrumentation to an aspect of the vertebral column that is displaced from the straddled aspect of the vertebral column. The surgical instrumentation guide 40 may also be configured to retract tissue by extending an attachment or sleeve 42. This attachment or sleeve 42 is typically adjustably coupled to the surgical instrument guide 40 such that the attachment or sleeve 42 may laterally extend from the surgical instrument guide 40 so as to retract tissue from the straddled aspects and the displaced aspects of the vertebral column. In addition, the spinal docking system 10 may comprise another attachment or sleeve 42 that may be, but is not necessarily, adjustably coupled to the spinal docking device 20. This additional attachment or sleeve 42 may laterally extend in a direction opposite from, or perpendicular to, the attachment or sleeve 42 adjustably coupled to the surgical instrument guide 40. For example, and not by way of limitation, the attachments or sleeves 42 may be configured as cylindrical retractors or rectangular retractor blocks that, when laterally expanded, form a boxed opening in the tissue with the surgical instrument guide 40 and attachments or sleeves 42 serving as the points of retraction that form this boxed opening in the tissue. An attachment or sleeve 42 may also be configured with a handle 60 so as to provide further stabilization and positioning. The handle may be configured in a variety of shapes so as not to impede the passage of surgical instrumentation into the surgical instrument guide 40.

In addition, the surgical instrument guide 40 may be designed in different lengths or configured to adjust in length along the longitudinal axis. Furthermore, the surgical instrumentation guide 40 may be configured to allow endoscopic visualization by guiding a surgical scope to the displaced aspect of the vertebral column. For example, and not by way of limitation, this surgical instrumentation guide 40 can be configured as a tube, a portion of a tube, or as a groove formed in a guide. The surgical instrumentation guide 40 may be configured to comprise one or more secondary tubes, portions of tubes, grooves, or any combination thereof, that are secured to surgical instrumentation guide 40. These secondary tubes, portions of tubes, or grooves may comprise central ports, side ports, or both, to provide various avenues for introduction of surgical instrumentation into the surgical instrument guide 40. This surgical instrumentation guide 40 is generally constructed of a rigid material, a flexible material, or a combination thereof. Such materials include, but are not limited to, surgical grade stainless steel or latex, or latex free, tubing. The spinal docking system 10 may further comprise a plurality of these surgical instrumentation guides 40. For example, a surgical instrument guide 40 may be coupled to two or more longitudinal faces 24 of the spinal docking device 20.

The spinal docking system 10 may further comprise a position-measuring indicator 50 that provides a visual representation of the position of the spinal docking device 20 in relation to the straddled aspect of the vertebral column or to a plumb line, perpendicular to the line of gravity. This position-measuring indicator 50 is generally rotatably secured to the spinal docking device 20 at an end distal from the straddled aspect of the vertebral column. This rotatability of the indicator 50 allows, among other things, for surgical instrumentation to more easily access the opening of the surgical instrument guide distal from the displaced aspect of the vertebral column. The indicator 50 typically comprises a mechanical, electronic, gravitational, computerized, or any combination thereof, position-measuring device. In addition, the spinal docking system 10 may further comprise a position-measuring indicator 50 that provides a visual representation of the position of the surgical instrumentation guide 40 in relation to the spinal docking device 20 or to the displaced aspect of the vertebral column, or both. Likewise, this indicator 50 typically is rotatably secured to the surgical instrumentation guide 40 at an end distal from the displaced aspect of the vertebral column. Alternatively, the spinal docking system 10 may comprise one or more position-measuring indicators 50 that provide visual representations of the positions of both the spinal docking device 20 and the surgical instrumentation guide 40.

In addition, the spinal docking system 10 may further comprise a surgical instrument positioning indicator that provides a visual representation of the position of a surgical instrument in relation to aspects of the vertebral column. This surgical instrument positioning indicator may be, but is not limited to, a frameless, or stealth, navigation system. For example, this surgical instrument positioning indicator allows for the graphic visualization, through use of infrared imaging and a video display, of a surgical instrument as it is introduced to a transverse process 1.

The spinal docking system 10 generally further comprises a handle 60 for further stabilization or positioning purposes. The handle 60 may be configured in a variety of shapes so as not to impede the passage of surgical instrumentation into the surgical instrument guide 40. In addition, the spinal docking system may further comprise a light source 70 that is configured to illuminate the displaced aspect of the vertebral column or the straddled aspect of the vertebral column. This light source 70 may be a bulb, optical fiber, mirror, or any other source of illumination and may be affixed to the surgical instrument guide 40 or to the spinal docking device 20.

It is contemplated by the present invention that the spinal docking system 10 is configured so as to be operated percutaneously. In such use, the spinal docking device 20 and the surgical instrument guide 40 are introduced into the straddled aspect and the displaced aspect of the vertebral column through minimally invasive percutaneous incisions.

In another embodiment of the present invention, a spinal docking system 10 comprises a spinal docking device 20 and a surgical instrumentation guide 40. The spinal docking device 20 typically comprises a concave end 22 and one or more longitudinal faces 24. This concave end 22 comprises a degree of concavity characterized by dimensions of width and depth sufficient to allow the concave end 22 to straddle an aspect of the vertebral column having a lateral dimension at least about half as large as a lateral dimension of a transverse process 1 of the vertebral column. These longitudinal faces 24 typically are secured to the surgical instrumentation guide 40. This surgical instrumentation guide 40, meanwhile, generally is configured to guide surgical instrumentation to the straddled aspect of the vertebral column. Generally, the surgical instrumentation guide 40 is oriented parallel to the longitudinal axis of the spinal docking device 22 and may be either centered on or offset from this longitudinal axis.

In yet another embodiment of the present invention, a spinal docking device 20 comprises a concave end 22 and one or more longitudinal faces 24. The concave end 22 generally comprises a degree of concavity characterized by dimensions of width and depth sufficient to allow the concave end 22 to straddle an aspect of the vertebral column having a lateral dimension at least about half as large as a lateral dimension of a transverse process 1 of the vertebral column. The longitudinal face 24 typically comprises a channeled depression to guide surgical instrumentation to the straddled aspect of the vertebral column or to an aspect of the vertebral column that is displaced from the straddled aspect of the vertebral column. This channeled depression typically is provided in a curved, angular, multi-angular, or combination thereof, configuration. Furthermore, the channeled depression generally is oriented parallel to the longitudinal axis of said spinal docking device 20 and may be centered on or offset from this longitudinal axis.

In accordance with yet another embodiment of the present invention, a method of stabilizing a pedicle is presented. The method comprises providing a spinal docking system that comprises a spinal docking device, a docking device coupling mechanism, and a surgical instrumentation guide. This spinal docking device comprises a concave end with a degree of concavity that is characterized by dimensions of width and depth that are sufficient to allow the concave end to straddle a transverse process of a vertebral column. The method further comprises positioning the concave end and the surgical instrumentation guide, which is adjustably secured to the spinal docking device by the docking device coupling mechanism, on a transverse process such that the spinal docking device and the surgical instrumentation guide are aligned substantially perpendicularly to the transverse process. Thereafter, the method comprises guiding through the surgical instrumentation guide a surgical instrument in the general direction of the transverse process and operating the surgical instrument so as to enable the insertion of spinal stabilization hardware through the transverse process and into the pedicle. Such spinal stabilization hardware may be, but is not limited to, a pedicle screw.

It is contemplated by the present invention that all of the aforementioned components of all of the described embodiments may be constructed of a variety of materials. In addition, these components may comprise radio-opaque, radio-lucent, or magneto-opaque, or any combination thereof, materials.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the invention in detail and by reference to specific embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

What is claimed is:

1. A spinal docking system comprising a spinal docking device, a docking device coupling mechanism, and a surgical instrumentation guide, wherein:
said spinal docking device comprises a concave end;
said concave end comprises a degree of concavity characterized by dimensions of width and depth sufficient to allow said concave end to straddle an aspect of the vertebral column having a lateral dimension at least about half as large as a lateral dimension of a transverse process of said vertebral column;
said docking device coupling mechanism is configured to secure said spinal docking device to said surgical instrumentation guide;
said docking device coupling mechanism is configured to allow movement of said surgical instrumentation guide relative to said spinal docking device; and
said surgical instrumentation guide is configured to guide surgical instrumentation to an aspect of the vertebral column displaced from said straddled aspect of the vertebral column.

2. The spinal docking system of claim 1, wherein said spinal docking device, said docking device coupling mechanism, and said surgical instrumentation guide are configured such that said straddled aspect and said displaced aspect comprise respective portions of a common aspect of the vertebral column.

3. The spinal docking system of claim 2, wherein said straddled aspect and said displaced aspect comprise respective portions of a transverse process of the vertebral column.

4. The spinal docking system of claim 1, wherein said spinal docking device, said docking device coupling mechanism, and said surgical instrumentation guide are configured such that said straddled aspect and said displaced aspect comprise different aspects of the vertebral column.

5. The spinal docking system of claim 4, wherein said straddled aspect comprises a portion of a transverse process and said displaced aspect comprises a portion of an adjacent facet and pedicle complex.

6. The spinal docking system of claim 4, wherein said straddled aspect comprises a portion of a pedicle, transverse process, facet, spinous process, or other spinal structure of the vertebral column and said displaced aspect comprises a portion of a pedicle, transverse process, facet, spinous process, or other spinal structure of the vertebral column different from the portion comprised by said straddled aspect.

7. The spinal docking system of claim 1, wherein said concave end is provided in a curved, angular, multi-angular, or combination thereof, configuration.

8. The spinal docking system of claim 1, wherein said spinal docking device is configured with a mechanism to vary the depth of said concave end.

9. The spinal docking system of claim 1, wherein said spinal docking device is configured with a mechanism to vary the width of said concave end.

10. The spinal docking system of claim 1, wherein said concave end is provided with clamping hardware configured to clamp said concave end onto said straddled aspect of the vertebral column.

11. The spinal docking system of claim 1, wherein said spinal docking device is configured such that said concave end is offset from a longitudinal axis of said spinal docking device.

12. The spinal docking system of claim 1, wherein said docking device coupling mechanism comprises a device or assembly configured to allow movement of said surgical instrumentation guide relative to said spinal docking device in a plurality of degrees of freedom.

13. The spinal docking system of claim 1, wherein said docking device coupling mechanism comprises hardware configured to indicate the positioning of said surgical instrumentation guide relative to said spinal docking device.

14. The spinal docking system of claim 1, wherein said docking device coupling mechanism comprises hardware configured to indicate the positioning of said surgical instrumentation guide relative to a plumb line and the line of gravity.

15. The spinal docking system of claim 1, wherein said spinal docking device comprises a longitudinal face configured to define a vertical slot to allow movement of said surgical instrumentation guide along said longitudinal face.

16. The spinal docking system of claim 1, wherein said surgical instrumentation guide is configured to retract tissue.

17. The spinal docking system of claim 16, wherein said surgical instrumentation guide comprises a laterally expandable attachment or sleeve to retract tissue.

18. The spinal docking system of claim 1, wherein said surgical instrument guide is configured to adjust in length along the longitudinal axis.

19. The spinal docking system of claim 1, wherein said surgical instrumentation guide is configured as a tube, a portion of a tube, or as a groove formed in a guide.

20. The spinal docking system of claim 19, wherein said surgical instrumentation guide comprises one or more secondary tubes, portions of tubes, grooves, or any combination thereof, secured to said surgical instrumentation guide.

21. The spinal docking system of claim 1, wherein said spinal docking system further comprises a position-measuring indicator that provides a visual representation of the position of said spinal docking device in relation to said straddled aspect of the vertebral column.

22. The spinal docking system of claim 1, wherein said spinal docking system further comprises a position-measuring indicator that provides a visual representation of the position of said surgical instrumentation guide in relation to said spinal docking device or to said displaced aspect of the vertebral column, or both.

23. The spinal docking system of claim 1, wherein said spinal docking system further comprises one or more position-measuring indicators that provide visual representations of the positions of both said spinal docking device and said surgical instrumentation guide.

24. The spinal docking system of claim 1, wherein said spinal docking system further comprises a surgical instrument positioning indicator that provides a visual representation of the position of a surgical instrument in relation to aspects of the vertebral column.

25. The spinal docking system of claim 1, wherein said spinal docking system further comprises a handle for further stabilization or positioning purposes.

26. The spinal docking system of claim 1, wherein said spinal docking system comprises a light source configured to illuminate said displaced aspect of the vertebral column or said straddled aspect of said vertebral column.

27. A spinal docking system comprising a spinal docking device and a surgical instrumentation guide, wherein:
said spinal docking device comprises a concave end and one or more longitudinal faces;
said concave end comprises a degree of concavity characterized by dimensions of width and depth sufficient to allow said concave end to straddle an aspect of the vertebral column having a lateral dimension at least about half as large as a lateral dimension of a transverse process of said vertebral column;
said one or more longitudinal faces are secured to said surgical instrumentation guide; and
said surgical instrumentation guide is configured to guide surgical instrumentation to said straddled aspect of the vertebral column.

28. The spinal docking system of claim 27, wherein said surgical instrumentation guide is oriented parallel to and offset from the longitudinal axis of said spinal docking device.

29. A spinal docking device comprising a concave end and one or more longitudinal faces, wherein:
said concave end comprises a degree of concavity characterized by dimensions of width and depth sufficient to allow said concave end to straddle an aspect of the vertebral column having a lateral dimension at least about half as large as a lateral dimension of a transverse process of said vertebral column; and
said longitudinal face comprises a channeled depression to guide surgical instrumentation to said straddled aspect of the vertebral column or to an aspect of the vertebral column displaced from said straddled aspect of the vertebral column.

30. The spinal docking device of claim 29, wherein said channeled depression is provided in a curved, angular, multi-angular, or combination thereof, configuration.

31. The spinal docking device of claim 29, wherein said channeled depression is oriented parallel to and offset from the longitudinal axis of said spinal docking device.

32. A method of stabilizing a pedicle comprising:
providing a spinal docking system comprising a spinal docking device, a docking device coupling mechanism, and a surgical instrumentation guide, wherein said spinal docking device comprises a concave end with a degree of concavity characterized by dimensions of width and depth sufficient to allow said concave end to straddle a transverse process of a vertebral column;
positioning said concave end on said transverse process such that said spinal docking device is aligned substantially perpendicularly to said transverse process;
aligning said surgical instrumentation guide adjustably secured to said spinal docking device by said docking device coupling mechanism with said spinal docking device so that said surgical instrumentation guide is substantially perpendicularly aligned to said transverse process;
guiding through said surgical instrumentation guide a surgical instrument in the general direction of said transverse process; and
operating said surgical instrument so as to enable insertion of spinal stabilization hardware through said transverse process and into said pedicle.

33. The method of claim 32, wherein said spinal stabilization hardware comprises a pedicle screw.
